(19) United States
(12) Patent Application Publication
(10) Pub. No.: US 2015/0164447 A1
(43) Pub. Date: Jun. 18, 2015

(54) METHOD AND SYSTEM FOR INTEGRATED MEDICAL TRANSPORT BACKBOARD DIGITAL X-RAY IMAGING DETECTOR

(71) Applicant: General Electric Company, Schenectady, NY (US)

(72) Inventors: Aaron Judy Couture, Schenectady, NY (US); Andrea Marie Schmitz, Niskayuna, NY (US); John Edward Close, Brookfield, WI (US)

(73) Assignee: General Electric Company, Schenectady, NY (US)

(21) Appl. No.: 14/109,454

(22) Filed: Dec. 17, 2013

(51) Int. Cl. A61B 6/00; A61G 1/003

(52) U.S. Cl. CPC A61B 6/42; A61G 1/003

(57) ABSTRACT

A method of imaging a patient and an X-ray imaging system are provided. The X-ray imaging system includes a support platform configured to support an object to be imaged and a digital X-ray imaging detector configured to receive incident radiation that has passed through the object, the X-ray imaging detector including a flexibility that permits the X-ray imaging detector to conform to a surface of the support platform, the X-ray imaging detector including a thickness of less than about four millimeters.

### BACKGROUND

This description relates to radiation imaging detectors, and, more particularly, to a system and method for imaging a patient directly on a patient transport backboard using a digital X-ray imaging detector.

At least some known digital X-ray (DXR) imaging detectors are fabricated on thick glass substrates. The glass substrate requires significant thickness and weight for packaging required to protect the substrate from breaking during use, transportation and storage. A critical limitation for a highly portable, front-line deployed digital X-ray imaging detector is the glass substrate.

The fragile glass substrate also dictates that current portable products have a limited ruggedness specification, including, for example, a maximum 30 centimeter (cm) drop height. The fragile substrate dictates the need for a heavy, thick, and stiff detector package. The thick cases make the devices difficult to incorporate into existing hospital or medical infrastructure and trade-offs are required to balance detector ruggedness against detector weight and thickness.

### BRIEF DESCRIPTION

In one embodiment, an X-ray imaging system includes a support platform configured to support an object to be imaged and a digital X-ray imaging detector configured to receive incident radiation that has passed through the object, the X-ray imaging detector including a flexibility that permits the X-ray imaging detector to conform to a surface of the support platform, the X-ray imaging detector including a thickness of less than about four millimeters.

In another embodiment, a method of imaging a patient includes providing a patient transport backboard including a flexible substrate digital X-ray imaging detector coupled to a surface of the patient transport backboard, the X-ray imaging detector including a flexible substrate, a thin film transistor (TFT) array, a photosensor layer, and a flexible scintillator and positioning a patient on the backboard with a portion of the patient to be imaged located adjacent to digital X-ray imaging detector.

In yet another embodiment, an emergency medical services backboard system includes a support platform configured to support a human patient to be imaged and a digital X-ray imaging detector configured to receive incident radiation that has passed through the patient forming at least a portion of the digital X-ray imaging detector, the digital X-ray imaging detector including a substrate, a flexible thin film transistor (TFT) array, a flexible photosensor layer, and a flexible scintillator layer, the digital X-ray imaging detector including a thickness of less than about four millimeters.

### DRAWINGS

FIGS. 1-4 show example embodiments of the method and apparatus described herein.

FIG. 1 is a schematic block diagram of an X-ray imaging system in accordance with an example embodiment of the present disclosure.

FIG. 2 is a perspective cut-away view of a physical arrangement of the components of an exemplary scintillation-based imaging detector suitable for use as imaging detector shown in FIG. 1.

FIG. 3 is a perspective view of a workpiece examination platform system in accordance with an example embodiment of the present disclosure.

FIG. 4 is a perspective view of a medical transport backboard system, also referred to as an emergency medical services (EMS) backboard in accordance with an example embodiment of the present disclosure.

Although specific features of various embodiments may be shown in some drawings and not in others, this is for convenience only. Any feature of any drawing may be referenced and/or claimed in combination with any feature of any other drawing.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

The following detailed description illustrates embodiments of the disclosure by way of example and not by way of limitation. It is contemplated that the disclosure has general application to structural and methodical embodiments of an X-ray imaging system having a robust digital X-ray imaging detector integrally formed in a patient transport backboard.

Embodiments of the present disclosure describe a flexible, unbreakable, thin substrate that enables a thin, rugged, light-weight X-ray imaging detector, also known as a photodetector. Flexible substrates permit a potential for new image acquisition and processing capability. In various embodiments, the substrate is composed of rigid or flexible materials such as glass, plastic, metals, or combinations thereof. For example, the substrate may include materials such as, but not limited to, glass, polyethylene terephthalate, polybutylene phthalate, polyethylene naphthalate, polystyrene, polycarbonate, polyether sulfone, polyallylate, polyimide, polycycloolefin, norbornene resins, fluropolymers, stainless steel, aluminum, silver, gold, and metal oxides (e.g., titanium oxide and zinc oxide), semiconductors (e.g., silicon or organic), or any other suitable material. Described herein is a portable X-ray imaging detector component that is integrated into an emergency medical service (EMS) backboard. EMS backboards are used to immobilize patients in the field that may have injuries which could be worsened with patient movement. The integrated X-ray imaging detector permits imaging of the patient in the field without patient movement. The integrated X-ray imaging detector enables rapid feedback on a severity of internal injuries of the patient while still located in the field. In addition, the backboard with the integrated portable X-ray imaging detector component remains a lightweight, rugged, and portable structure.

In an embodiment, the photodetector is fabricated over a pixel element array, also referred to as a thin film transistor (TFT) array, which is formed or positioned over a substrate. The photodetector is typically fabricated directly over the imaging TFT array. The photodetector, also referred to as a photodiode or an organic photodiode (OPD), may include an anode, a cathode, and an organic film between the anode and the cathode, which produces charged carriers in response to absorption of light. A scintillator may be formed or positioned over the cathode of the photodetector, and a top cover may cover the scintillator.

By using an unbreakable material instead of a fragile glass substrate for the X-ray imaging detector, the components and materials which are used in current imaging detectors to absorb bending stress or drop shock can be reduced in size and weight or eliminated. The overall weight and thickness of the digital X-ray imaging detector is able to be reduced and is conducive to integration in the EMS backboard.

By removing costly materials which are used to protect the glass substrate used in current X-ray imaging detectors, the overall cost of the digital X-ray imaging detector is decreased. In addition, the number of patterned layers needed for the digital X-ray imaging detector is reduced by utilizing an un-patterned, low cost organic photodiode. Both of these are advantages for the flexible substrate and organic photodiode.

The glass substrate used in currently available X-ray imaging detector is the single most breakable component in the detector. The glass substrate no longer being used in the digital X-ray imaging detector permits a larger drop height, detector patient loading weight, and overall ruggedness.

In various embodiments, a flexible substrate is be integrated into a rugged digital X-ray imaging detector composed of a flexible substrate, a TFT array, a high performance organic photodiode and flexible scintillator. The portable digital X-ray imaging detector is light-weight, rugged and flexible.

The following description refers to the accompanying drawings, in which, in the absence of a contrary representation, the same numbers in different drawings represent similar elements.

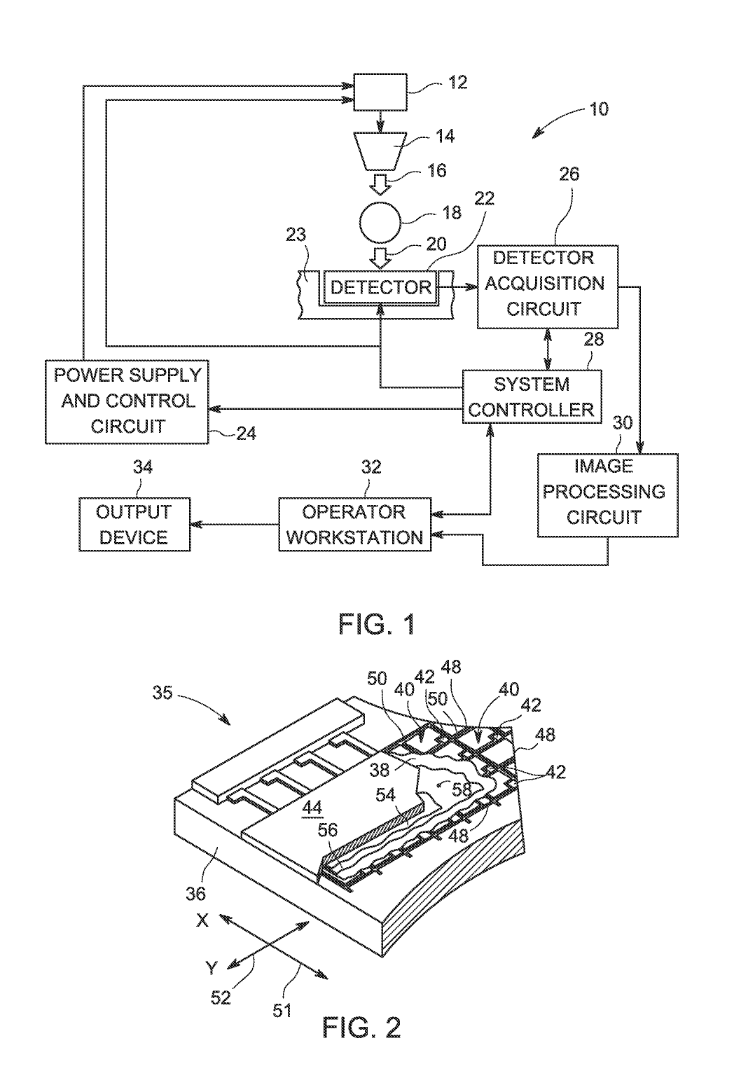

FIG. 1 is a schematic block diagram of an X-ray imaging system 10 in accordance with an example embodiment of the present disclosure. In the example embodiment, X-ray imaging system 10 is configured to acquire and process X-ray image data. X-ray imaging system 10 includes an X-ray source 12, a collimator 14, and an imaging detector 22. In one embodiment, imaging detector 22 is mounted on a support platform 23 by either coupling imaging detector 22 to a surface of support platform 23 or embedded in a well formed in the surface of support platform 23. In various embodiments, imaging detector 22 is embodied in a tethered detector, which may be positioned on support platform 23 at any location of interest between the surface of support platform 23 and a patient. X-ray source 12 can be positioned adjacent to the collimator 14. In one embodiment, X-ray source 12 is a low-energy source and is employed in low energy imaging techniques, such as, but not limited to, radiographic and fluoroscopic techniques. Collimator 14 can permit a stream of X-ray radiation 16 emitted by X-ray source 12 to radiate towards a target 18, such as an industrial component or a human patient. A portion of X-ray radiation 16 is attenuated by target 18 and at least some attenuated radiation 20 impacts imaging detector 22, such as a radiographic or fluoroscopic imaging detector.

Imaging detector 22 may be based on scintillation, i.e., optical conversion, direct conversion, or on other techniques used in the generation of electrical signals based on incident radiation. For example, a scintillator-based imaging detector converts X-ray photons incident on its surface to optical photons. These optical photons may then be converted to electrical signals by employing photosensor(s), e.g., photodiode(s). Conversely, a direct conversion imaging detector directly generates electrical charges in response to incident X-ray photons. The electrical charges can be stored and read out from storage capacitors. As described in detail below, these electrical signals, regardless of the conversion technique employed, are acquired and processed to construct an image of the features (e.g., anatomy) within target 18.

In the example embodiment, X-ray source 12 is controlled by a power supply and control circuit 24 which supplies power and control signals for examination sequences. In various embodiments, exposure timing is controlled automatically by an auto-sensor associated with X-ray source 12. In other embodiments, X-ray source 12 is embodied in one or more radioisotopes wherein power supply and control circuit 24 supplies power and control signals for examination sequences using the radioisotopes. Moreover, imaging detector 22 can be coupled to a detector acquisition circuit 26, which can be configured to receive electrical readout signals generated in imaging detector 22. Detector acquisition circuit 26 may also execute various signal processing and filtration functions, such as, for initial adjustment of dynamic ranges and interleaving of digital signals.

In the example embodiment, one or both of power supply/control circuit 24 and detector acquisition circuit 26 can be responsive to signals from a system controller 28. System controller 28 can include signal processing circuitry, typically based upon a general purpose or application specific digital computer programmed to process signals according to one or more parameters. System controller 28 may also include memory circuitry for storing programs and routines executed by the computer, as well as configuration parameters and image data and interface circuits.

System 10 can include an image processing circuit 30 configured to receive acquired projection data from detector acquisition circuit 26. Image processing circuit 30 can be configured to process the acquired data to generate one or more images based on X-ray attenuation.

An operator workstation 32 can be communicatively coupled to system controller 28 and/or image processing circuit 30 to allow an operator to initiate and configure X-ray imaging of target 18 and to view images generated from X-rays that impinge imaging detector 22. For example, system controller 28 is in communication with operator workstation 32 so that an operator, via one or more input devices associated with operator workstation 32, may provide instructions or commands to system controller 28. Operator workstation 32 represents various forms of digital computers, such as laptops, desktops, workstations, personal digital assistants, servers, tablets, and other appropriate computers. Operator workstation 32 is also intended to represent various forms of mobile devices, such as personal digital assistants, cellular telephones, smart phones, and other similar computing devices. The components shown here, their connections and relationships, and their functions, are meant to be examples only, and are not meant to limit implementations of the subject matter described and/or claimed in this document.

Similarly, image processing circuit 30 can be in communication with operator workstation 32 such that operator workstation 32 can receive and display the output of image processing circuit 30 on an output device 34, such as a display or printer. Output device 34 may include standard or special purpose computer monitors and associated processing circuitry. In general, displays, printers, operator workstations, and similar devices supplied within system 10 may be local to the data acquisition components or may be remote from these components, such as elsewhere within an institution or hospital or in an entirely different location. For example, system 10 may form a portion of an emergency response vehicle, such as, but not limited to an ambulance. During a field evaluation of a patient, prior to the patient arriving at a healthcare facility, system 10 may acquire images of the patient, and those images may be transmitted wirelessly to the healthcare facility. Output devices and operator workstations that are remote from the data acquisition components may be operatively coupled to the image acquisition system via one or more configurable networks, such as the Internet or virtual private networks. Though system controller 28, image processing circuit 30, and operator workstation 32 are shown distinct from one another in FIG. 1, these components may actually be embodied in a single processor-based computing system. Alternatively, some or all of these components may be present in distinct processor-based computing systems configured to communicate with one another. For example, image processing circuit 30 may be a component of a distinct reconstruction and viewing workstation.

FIG. 2 is a perspective cut-away view of a physical arrangement of the components of an exemplary scintillation-based imaging detector 35 suitable for use as imaging detector 22 depicted in FIG. 1. Imaging detector 35 can include a flexible substrate 36 upon which one or more components can be deposited. For example, in the present embodiment, imaging detector 35 can include a continuous photosensor element 38, transistors 42 (e.g., amorphous Silicon (a-Si) thin-film transistors (TFTs)), scintillator 44, data readout lines 48, scan lines 50, a conductive layer 54, and a dielectric layer 56 deposited with respect to substrate 36. Substrate 36 may be composed of rigid or flexible materials such as glass, plastic, metals, or combinations thereof. For example, substrate 36 may include materials such as, but not limited to, glass, polyethylene terephthalate, polybutylene phthalate, polyethylene naphthalate, polystyrene, polycarbonate, polyether sulfone, polyallylate, polyimide, polycycloolefin, norbornene resins, fluropolymers, stainless steel, aluminum, silver, gold, and metal oxides (e.g., titanium oxide and zinc oxide), semiconductors (e.g., silicon or organic), or any other suitable material. The components of imaging detector 35 can be composed of metallic, dielectric, organic, and/or inorganic materials, and can be fabricated with respect to substrate 36 using various material deposition and removal techniques. Some examples of deposition techniques include, for example, chemical vapor deposition, physical vapor deposition, electrochemical deposition, stamping, printing, sputtering, slot die coating, and/or any other suitable deposition technique. Some examples of material removal techniques include lithography, etching (e.g., dry, wet, laser), sputtering, and/or any other suitable material removal techniques.

Imaging detector 35 can include an array of pixel areas 40 on flexible substrate 36. Each of pixel areas 40 can include transistors 42 operatively coupled to respective data readout lines 48, scan lines 50, and photosensor 38. In the present embodiment, transistors 42 are arranged in a two dimensional array having rows extending along an x-axis 51 and columns extending along a y-axis 52, or vice versa. In some embodiments, transistors 42 can be arranged in other configurations. For example, in some embodiments, transistors 42 can be arranged in a honeycomb pattern. A spatial density of transistors 42 can determine a quantity of pixel areas 40 or pixels in the array, the physical dimensions of the array, as well as the pixel density or resolution of imaging detector 35.

Each of data readout lines 48 can be in electrical communication with an output of a respective transistor 42. For example, each of data readout lines 48 can be associated with a row or column of transistors 42, and the output (e.g., source or drain) of each transistor 42 in the row or column can be in electrical communication with the same data readout line 48 such that there is one data readout line per row or column. Data readout lines 48 are susceptible to interference, such as electronic noise from a surrounding environment, which can affect data signals being transmitted on data readout lines 48. Data readout lines 48 can be formed of a conductive material, such as a metal, and can be configured to facilitate transmission of electrical signals, corresponding to incident X-rays, to image processing circuitry (e.g., image processing circuit 30).

Scan lines 50 can be in electrical communication with inputs (e.g., gates) of transistors 42. For example, each of scan lines 50 can be associated with a row or column of transistors 42 and the input of each of transistors 42 in the same row or column can be in electrical communication with one of scan lines 50. Electrical signals transmitted on scan lines 50 can be used to control transistors 42 to output data on the transistor's output such that each transistor 42 connected to one of scans lines 50 are configured to output data concurrently and data from each transistor 42 connected to one of scan lines 50 flows through data readout lines 48 in parallel. In various embodiments, scan lines 50 and data readout lines 48 can extend perpendicularly to one another to form a grid. Scan lines 50 can be formed of a conductive material, such as a metal, and can be configured to facilitate transmission of electrical signals from a controller (e.g., system controller 28) to an input of transistors 42.

Continuous photosensor 38 can be deposited over transistors 42, data readout lines 48, and/or scan lines 50. Photosensor 38 can be formed from one or more photoelectric materials, such as one or more organic (i.e., carbon-based) and/or inorganic (i.e., non-carbon-based) materials that that convert light into electric current. In the present embodiment, the photoelectric material can extend continuously as a unitary structure over the array of transistors 42, data readout lines 48, and scan lines 50 such that the photoelectric material of photosensor 38 substantially overlays and/or covers pixel areas 40. By using a continuous unpatterned photoelectric material that is disposed over the transistor array, the density of transistors 42 in the array, and therefore, the pixel density of the imaging detector, can be increased as compared to patterned photosensors and/or a complexity of imaging detector fabrication can be reduced.

Electrodes (e.g., electrical contacts) of photosensor 38 can define anode(s) and cathode(s) of photosensor 38 and can be formed of a conductive material, such as, for example, indium tin oxide (ITO). For example, photosensor 38 can include electrodes disposed on a first side of photosensor 38 for electrically coupling the first side of photosensor 38 to transistors 42 and can include one or more electrodes disposed on a second opposing side of photosensor 38 for electrically coupling the second side of photosensor 38 to a bias voltage or vice versa. The electrodes of photosensor 38 can form the anode(s) or cathode(s) of photosensor 38.

A dielectric layer 56 can be disposed over continuous photosensor 38 and a conductive layer 54 can be disposed on dielectric layer 56. Dielectric layer 56 can include vias 58 to electrically couple conductive layer 54 to the electrode(s) of photosensor 38 to allow a common bias voltage to be applied at each pixel area 40 of imaging detector 35.

Scintillator 44 is disposed over conductive layer 54 and generates the optical photons when exposed to X-rays. The optical photons emitted by scintillator 44 are detected by photosensor 38, which converts the optical photons to an electrical charge that can be output through transistors 42 to data readout lines 48.

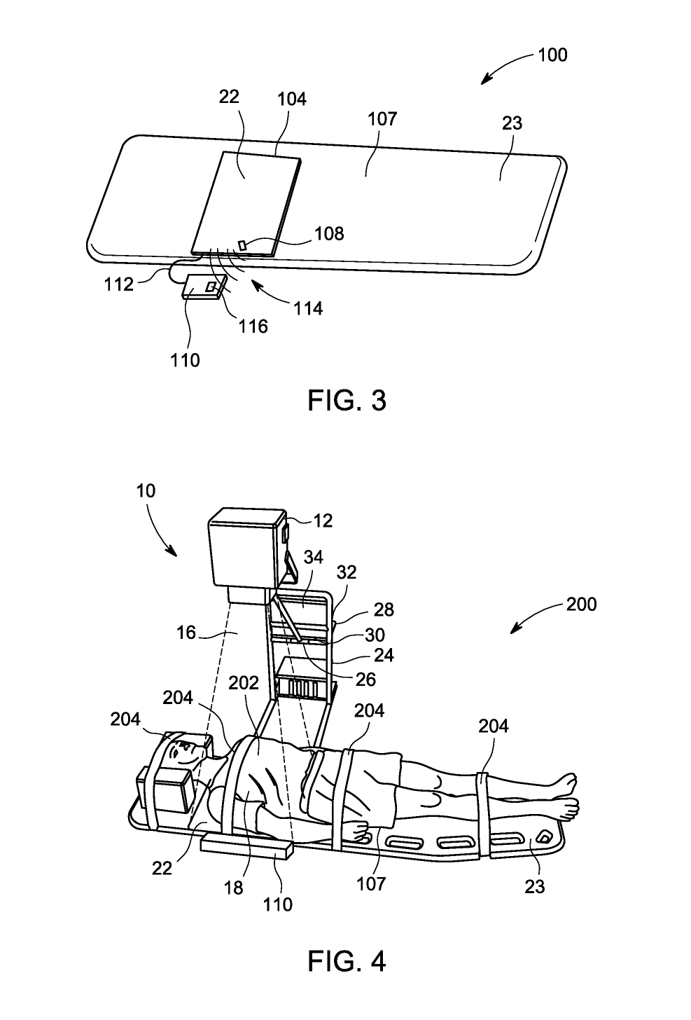

FIG. 3 is a perspective view of a workpiece examination platform system 100 in accordance with an example embodiment of the present disclosure. In the example embodiment, system 100 includes a support platform 23 configured to receive a workpiece or body (not shown in FIG. 1) to be examined. In one embodiment, the workpiece is an industrial component subject to an inspection using X-rays. In various embodiments, the workpiece may be an animal body, such as a human, which will undergo an X-ray examination when positioned on support platform 23. Support platform 23 may include a well 104 configured to receive X-ray imaging detector 22 therein. Well 104 may be embodied as a depression in support platform 23 that is sized to receive X-ray imaging detector 22. In one embodiment, well 104 is approximately four millimeters (mm) deep to accommodate a thickness of X-ray imaging detector 22 of approximately one-eighth of an inch (3.175 mm). In various other embodiments, well 104 is less deep to accommodate a thickness of X-ray imaging detector 22 of approximately one mm. X-ray imaging detector 22 may be formed separately from support platform 23 and subsequently coupled to support platform 23. Alternatively, X-ray imaging detector 22 may be formed in place on support platform 23. Support platform 23 may not include well 104, in other embodiments, but, rather, X-ray imaging detector 22 may be coupled to or formed directly on a surface 107 of support platform 23.

Signals representing an amount of X-rays received by pixel areas 40 (shown in FIG. 1) may be received by an onboard multiplexer 108 for transmission to an offboard controller 110. In one embodiment, the transmission is through a wired connection 112 to offboard controller 110. In other embodiments, the transmission is through a wireless connection 114 to a receiver 116 of offboard controller 110.

FIG. 4 is a perspective view of a medical transport backboard system 200, also referred to as an emergency medical services (EMS) backboard in accordance with an example embodiment of the present disclosure. EMS backboard system 200 includes support platform 23 embodied as an EMS backboard and imaging detector 22. EMS backboard system 200 is configured to be operable with system 10 to acquire, process, and output images of a human target 18. In the example embodiment, imaging detector 22 is located in an area proximate a chest 202 of human target 18. In various embodiments, imaging detector 22 is located in an area proximate other parts of a body of human target 18, such as, but not limited to, a head, a neck, a spine, a leg, and a foot of human target 18. Each of the various imaging detectors 22 may be connected to system controller 28, image processing circuit 30, and/or operator workstation 32 individually only when needed for that particular area. Alternatively, each of the various imaging detectors 22 may be permanently connected to system controller 28, image processing circuit 30, and/or operator workstation 32 and selected for use when needed electronically through system controller 28, image processing circuit 30, and/or operator workstation 32. Imaging detector 22 may be formed to cover an entirety of surface 107 of support platform 23 to make a full-body image for rapid triage diagnostics.

During operation, human target 18 is positioned on EMS backboard system 200 and secured using, for example, straps 204. System 10 is positioned adjacent EMS backboard system 200 and human target 18. One or more images of human target 18 are acquired and output locally and/or transmitted to selectable recipients.

It will be appreciated that the above embodiments that have been described in particular detail are merely example or possible embodiments, and that there are many other combinations, additions, or alternatives that may be included.

Also, the particular naming of the components, capitalization of terms, the attributes, data structures, or any other programming or structural aspect is not mandatory or significant, and the mechanisms that implement the disclosure or its features may have different names, formats, or protocols. Further, the system may be implemented via a combination of hardware and software, as described, or entirely in hardware elements. Also, the particular division of functionality between the various system components described herein is merely one example, and not mandatory; functions performed by a single system component may instead be performed by multiple components, and functions performed by multiple components may instead performed by a single component.

Some portions of above description present features in terms of algorithms and symbolic representations of operations on information. These algorithmic descriptions and representations may be used by those skilled in the data processing arts to most effectively convey the substance of their work to others skilled in the art. These operations, while described functionally or logically, are understood to be implemented by computer programs. Furthermore, it has also proven convenient at times, to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or “providing” or the like, refer to the action and processes of a computer system, or similar electronic computing device, that manipulates and transforms data represented as physical (electronic) quantities within the computer system memories or registers or other such information storage, transmission or display devices.

Based on the foregoing specification, the above-discussed embodiments of the disclosure may be implemented using computer programming or engineering techniques including computer software, firmware, hardware or any combination or subset thereof. Any such resulting program, having computer-readable and/or computer-executable instructions, may be embodied or provided within one or more computer-readable media, thereby making a computer program product, i.e., an article of manufacture, according to the discussed embodiments of the disclosure. The computer readable media may be, for instance, a fixed (hard) drive, diskette, optical disk, magnetic tape, semiconductor memory such as read-only memory (ROM) or flash memory, etc., or any transmitting/receiving medium such as the Internet or other communication network or link. The article of manufacture containing the computer code may be made and/or used by executing the instructions directly from one medium, by copying the code from one medium to another medium, or by transmitting the code over a network.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here, and throughout the specification and claims, range limitations may be combined and/or interchanged, such ranges are identified and include all the sub-ranges contained therein unless context or language indicates otherwise.

While the disclosure has been described in terms of various specific embodiments, it will be recognized that the disclosure can be practiced with modification within the spirit and scope of the claims.

The above-described embodiments of a system and method of imaging a workpiece provides a cost-effective and reliable means for positioning a workpiece, such as, but not limited to, a human patient on a backboard having an X-ray imaging detector built-in to a surface of the backboard. As a result, the system and method described herein facilitate imaging a patient proximate a site of injury using a robust imaging detector in a cost-effective and reliable manner.

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An X-ray imaging system comprising:
a support platform configured to support an object to be imaged;
a digital X-ray imaging detector configured to receive incident radiation that has passed through the object, the X-ray imaging detector comprising a flexibility that permits the X-ray imaging detector to conform to a surface of the support platform, the X-ray imaging detector comprising a thickness of less than about four millimeters.

2. The system of claim 1, wherein the X-ray imaging detector comprises a thickness of less than about two millimeters.

3. The system of claim 1, wherein the support platform includes a depression formed in the surface, the depression sized complementary to a size of the X-ray imaging detector.

4. The system of claim 1, wherein the X-ray imaging detector is adhesively coupled to the surface.

5. The system of claim 1, wherein the X-ray imaging detector is formed integrally on the surface.

6. The system of claim 1, wherein the support platform comprises a medical transport backboard.

7. The system of claim 1, wherein the digital X-ray imaging detector comprises a plurality of flexible layers including a flexible substrate, a thin film transistor (TFT) array, an organic photodiode (OPD) layer, and a flexible scintillator.

8. A method of imaging a patient comprising:
providing a patient transport backboard including a flexible substrate digital X-ray imaging detector coupled to a surface of the patient transport backboard, the X-ray imaging detector comprising a flexible substrate, a thin film transistor (TFT) array, a photosensor layer, and a flexible scintillator; and
positioning a patient on the backboard with a portion of the patient to be imaged located adjacent the digital X-ray imaging detector.

9. The method of claim 8, wherein positioning a patient on the backboard comprises positioning the patient on the backboard between the X-ray imaging detector and an X-ray source.

10. The method of claim 8, wherein providing a patient transport backboard comprises forming a well in an upper surface of the backboard, the well configured to receive the digital X-ray imaging detector.

11. The method of claim 10, wherein providing a patient transport backboard comprises forming the well with a depth below the surface approximately equal to a thickness of the digital X-ray imaging detector.

12. The method of claim 10, wherein providing a patient transport backboard comprises forming the well with a depth below the surface of less than approximately four millimeters (mm).

13. The method of claim 10, wherein providing a patient transport backboard comprises forming the well with a depth below the surface of approximately one millimeter (mm).

14. The method of claim 8, wherein providing a patient transport backboard comprises forming the digital X-ray imaging detector integrally with the backboard.

15. The method of claim 8, wherein providing a patient transport backboard comprises forming the digital X-ray imaging detector using the surface of the backboard as the substrate of the X-ray imaging detector.

16. The method of claim 8, wherein providing a patient transport backboard comprises forming the digital X-ray imaging detector using a flexible organic film substrate.

17. An emergency medical services backboard system comprising:
a support platform configured to support a human patient to be imaged; and
a digital X-ray imaging detector configured to receive incident radiation that has passed through the patient forming at least a portion of the digital X-ray imaging detector, the digital X-ray imaging detector comprising a substrate, a flexible thin film transistor (TFT) array, a flexible photosensor layer, and a flexible scintillator layer, the digital X-ray imaging detector comprising a thickness of less than about four millimeters.

18. The system of claim 17, wherein the digital X-ray imaging detector comprises a thickness of less than about two millimeters.

19. The system of claim 17, wherein the digital X-ray imaging detector comprises a thickness of less than about one millimeter.

20. The system of claim 17, wherein the support platform forms the substrate.
